(19) United States
(12) Patent Application Publication
(10) Pub. No.: US 2003/0105464 A1
(43) Pub. Date: Jun. 5, 2003

(54) Connecting body for bone pieces to be connected

(76) Inventors: Albert Wilhelm Schreurs, Leiden (NL); Simon Dante Strackee, Almere (NL)

Correspondence Address: PEACOCK MYERS AND ADAMS P C, P O BOX 26927, ALBUQUERQUE, NM, 871256927

(21) Appl. No.: 10/255,657

(22) Filed: Sep. 25, 2002

Related U.S. Application Data
(63) Continuation of application No. PCT/NL01/00260, filed on Mar. 29, 2001.

(30) Foreign Application Priority Data
Mar. 30, 2000 (CH) 1014790

(51) Int. Cl. A61B 17/58

(52) U.S. Cl. 606/72

(57) ABSTRACT

A connecting body for bone pieces to be joined together, characterized in that it has turned ends at both sides of a substantially elongated which may be completely driven into the bone pieces to be joined. At least one side of the body between the ends is preferably provided with a sharp edge. The body is preferably provided with at least one tooth pointing sideways and projecting away from the sharp edge.

The invention relates to a connecting body for bone pieces to be joined together.

Such a connecting body is known, for example, in the form of a staple or screws. Especially with stabilizing wrists the known connecting body exhibits disadvantages that necessitate a major operation in order for the desired stabilization to be carried out. To do this, the topside of the wrist joint has to be completely laid open and the various joint parts are completely stabilized. This is partly due to the fact that after the operation considerable forces may be exerted on the wrist that cannot be absorbed if a more limited operation is carried out. Such wrist stabilization in accordance with the prior art therefore is a major procedure, not only in the operative sense, but also with respect to the final use the stabilized wrist is capable of.

It is the object of the invention to provide a connecting body by means of which the operation may be simplified and by means of which there is a greater measure of remaining use for the operated wrist. It is also an object of the invention to provide a connecting body by means of which bone pieces generally may be joined in a manner that is reliable, solid and permanent.

To this end the connecting body according to the invention is characterized in that it has turned ends at both sides of a substantially elongated, possibly square body, which may be completely driven into the bone pieces to be joined. Preferably at least one side of the body between the ends is provided with a sharp edge.

The invention is also embodied in a method wherein such a connecting body is used for joining bone pieces. In accordance with this invention said method is characterized in that the body just mentioned is completely driven into the bone pieces to be joined. As a result, the connecting body is able to absorb all the forces occurring in practice, such as to provide the desired stable connection between the bone pieces. To do this, the turned ends at both sides of the body are obviously of importance.

This connecting body may be driven into the bone pieces to be joined in any suitably applied technique, such as preferably the techniques comprised in the group of hammering in, casu quo high-frequency or ultrasonic vibration.

An important advantage provided by the connecting body according to the invention is that the total volume of said connecting body is limited, so that devasculation in the bone is kept to a limit. The connecting body may be driven into the bone to under the cartilage surface, allowing it to be placed on the mechanically most favourable site without at the same time limiting the movability.

In contrast with the connecting bodies of the prior art such as screws and k-wires, the connecting body, thanks to the elongated shape, is not only very resistant to shearing forces but also to torsional forces. Especially when stabilizing wrist joint parts, it is possible to drive in laterally, with the result that the amount of surgery involved is greatly reduced and can be carried out practically with a minimum of invasion, so that the post-operative period of recovery may be considerably shortened, while the functionality of the wrist joint is maintained at a higher level than compared with the prior art.

In a preferred embodiment, the connecting body according to the invention is characterized in that the body is provided with at least one tooth pointing sideways and projecting away from the sharp edge. This ensures that after the body has been driven into the bone pieces to be joined, it is secured so that there is no danger that after some time the connecting body may be pushed out of the bone.

A simple embodiment, with which this may be realized, is characterized in that the tooth is pressed out from the surface of the body.

To ensure that the connecting body according to the invention is properly integrated in the bone pieces, it is desirable for the body to be provided with one or more perforations.

It is further preferred that the body be provided with a biocompatible coating. This not only aids the connecting body's growth into the bone pieces to be joined but also at the same time, due to its lubricating effect, helps when initially inserting the connecting body into the bone pieces to be joined.

In a further aspect of the invention, the connecting body is characterized in that the body is shaped like a C or an S. This design of the connecting bodies, together with the sharp edge provided at one side of the connecting body, affords the advantage that as a result of being driven into the bone pieces, the body undergoes a certain amount of stretching that exerts pressure on the bone pieces to be joined. This pressure may be pre-set by already slightly bending the connecting body open prior to or during the connecting body being driven in.

In another aspect of the invention the connecting body is characterized in that the body has a saw-tooth or waveform. By this means it is possible to realize a very reliable and solid attachment with the bone pieces, capable of absorbing considerable tensile forces.

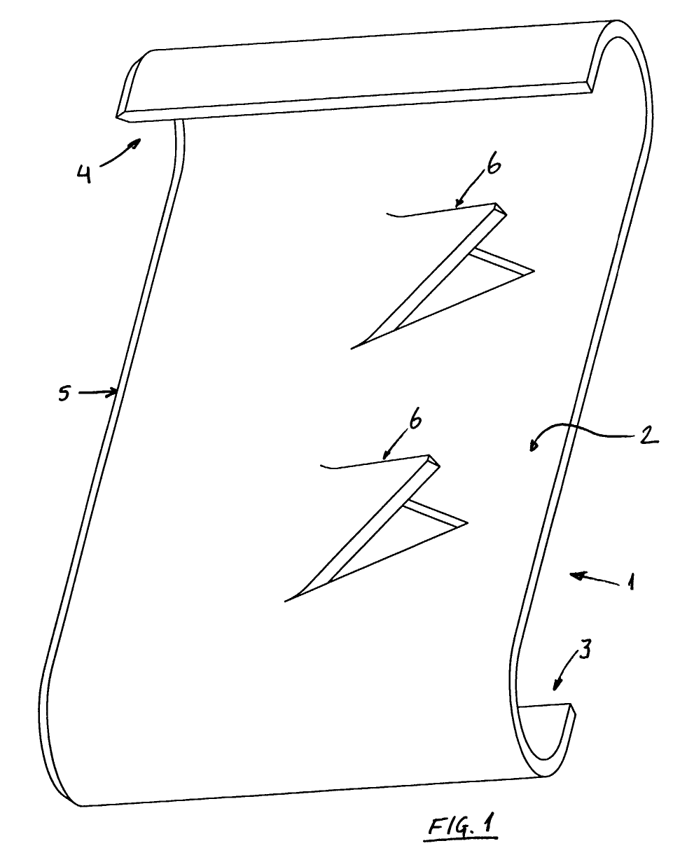

The invention will now be elucidated with reference to the drawing, which in the FIGS. 1 to 3 shows three different embodiment variants of the connecting body according to the invention.

It is explicitly remarked that the invention is not limited to the embodiment variants shown here, but that within the framework of the invention variations are possible, as long as they fall within the specifications of the appended claims.

Similar components in the figures are identified by the same reference numbers.

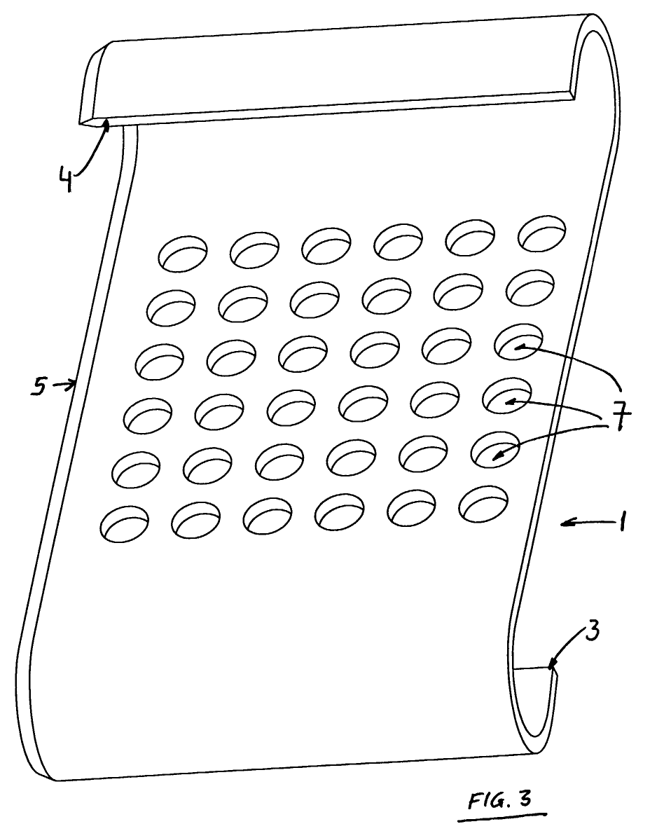

As is shown in the FIGS. 1 to 7, the connecting body 1 is embodied having a substantially elongated, optionally square, body 2 to be completely driven into the bone pieces (not shown). At both sides, the body 2 has turned ends 3 and 4. The embodiment variants shown in the FIGS. 1 to 7 additionally have a sharp edge 5 provided on the body 2 between the ends 3 and 4. The body 2 may further be provided with a tooth 6 pressed out from the surface of the body, preferably pointing in the direction opposite to the direction into which the sharp edge 5 is pointing. This is shown in FIG. 1. FIG. 3 shows the embodiment in which the body 2 is provided with one or more perforations 7. Further, the body 2 may be provided with a biocompatible coating.

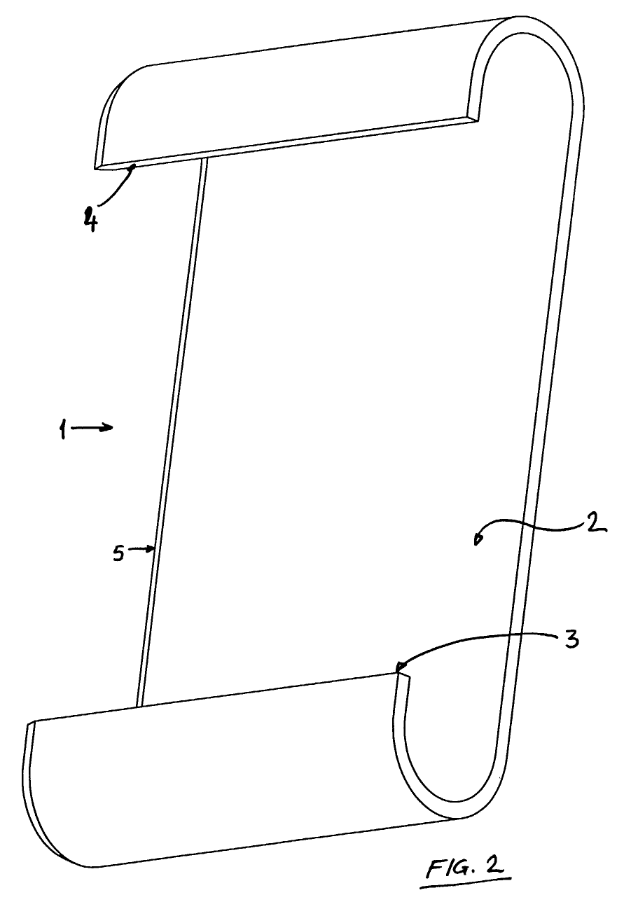

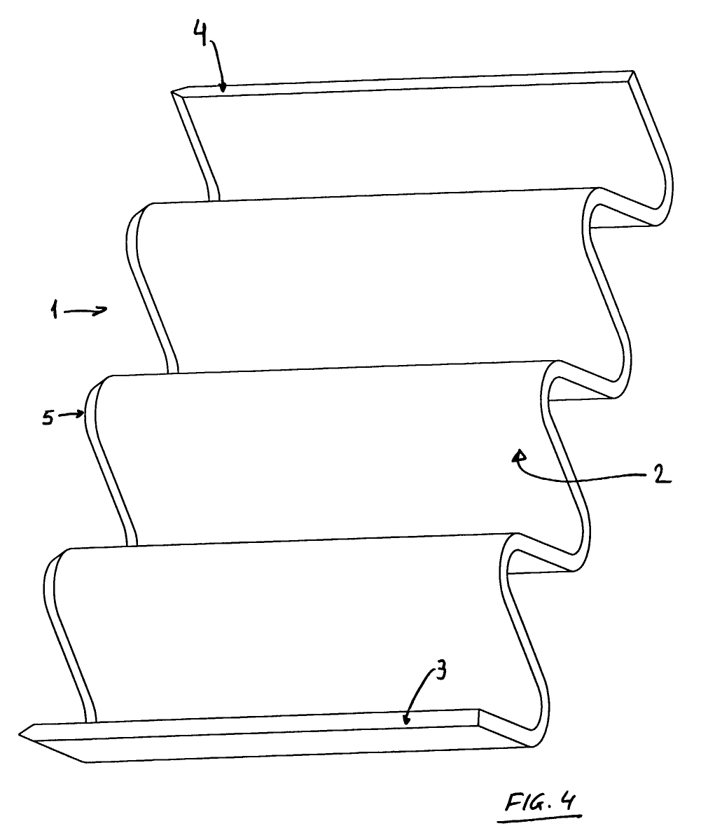

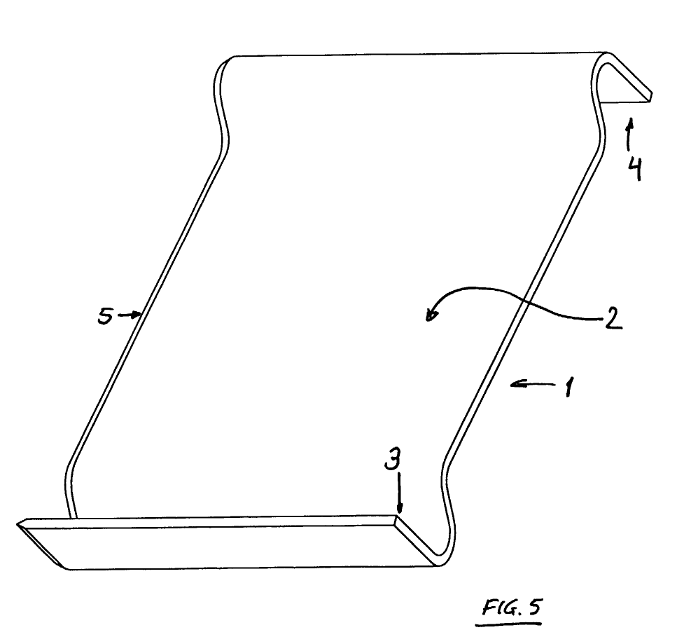

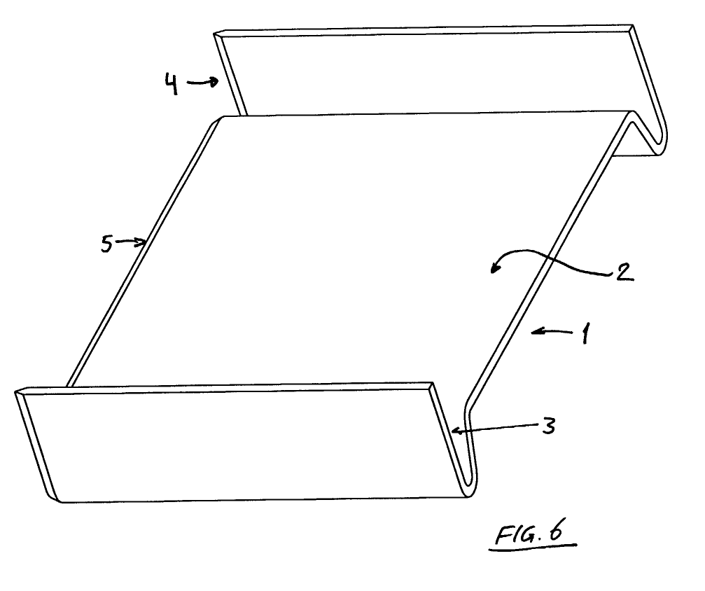

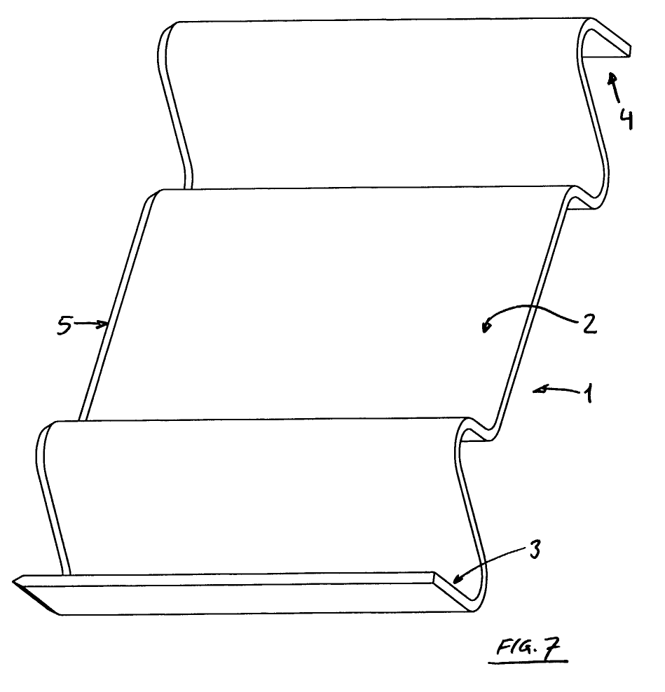

The embodiment variants shown in the figures are distinguished by the fact that the embodiment shown in FIGS. 1 and 3 are shaped like an S, the embodiment shown in FIG. 2 is shaped like a C, while the embodiment shown in FIG. 4 has an undulating body 2. FIGS. 5 and 6, show variations on the embodiment shaped like an S and a C, respectively, while FIG. 7, relates to an intermediate form between the S or C shape and the completely undulating body 2 shown in FIG. 4.

What is claimed is:

1. A connecting body for bone pieces to be joined together, said body comprising turned ends at both sides of a substantially elongated body, which ends are completely derivable into the bone pieces to be joined.

2. A connecting body according to claim 1, wherein at least one side of the body between the ends is provided with a sharp edge.

3. A connecting body according to claim 2, wherein the body is provided with at least one tooth pointing sideways and projecting away from the sharp edge.

4. A connecting body according to claim 3, wherein the tooth is pressed out from the surface of a body.

5. A connecting body according to claim 1, wherein the body is provided with one or more perforations.

6. A connecting body according to claim 1, wherein the body is provided with a biocompatible coating.

7. A connecting body according to claim 1, wherein the body is shaped like a C or an S.

8. A connecting body according to claim 1, wherein the body has a sawtooth or waveform.

9. A method of joining bone pieces, comprising driving the connecting body according to any of claims 1-8 completely into the bone pieces to be joined.

10. A method according to claim 9, wherein the connecting body is driven into the bone pieces by means of a technique chosen from the group consisting of hammering in and high-frequency casu quo ultrasonic vibration.
